(19) United States
(12) Patent Application Publication
(10) Pub. No.: US 2006/0194990 A1
(43) Pub. Date: Aug. 31, 2006

(54) Recycling method and system

(76) Inventors: Norihisa Miyoshi, Tokyo (JP); Takashi Imaizumi, Tokyo (JP); Yuki Iwadate, Tokyo (JP); Takao Kobayashi, Tokyo (JP); Seiichiro Toyoda, Tokyo (JP)

Correspondence Address: WENDEROTH, LIND & PONACK, L.L.P., 2033 K STREET N. W., SUITE 800, WASHINGTON, DC, 20006-1021, US

(21) Appl. No.: 10/566,673

(22) PCT Filed: Aug. 26, 2004
(86) PCT No.: PCT/JP04/12674

§ 371 Date: Feb. 1, 2006

(30) Foreign Application Priority Data
Aug. 29, 2003 (JP) 2003-307537
Jan. 7, 2004 (JP) 2004-002451

(51) Int. Cl. C10G 1/10; C07C 1/00; B01J 8/04

(52) U.S. Cl. 585/241; 422/188

(57) ABSTRACT

A recycling system has a pyrolysis apparatus (40) having a combustion section (42) for selectively combusting a carbon component in materials, and a gasification section (41) for pyrolyzing and gasifying the materials by using heat of combustion in the combustion section (42). The materials include residual oil from an atmospheric distillation unit (10), a vacuum distillation unit (20), and a decomposition unit (28), various wastes including waste plastics (181), wastes (182), and shredder dust (183), and organic matter such as biomass (184). The recycling system has a passage for supplying pyrolysates (120) produced in the gasification section (41) to an oil refinery system or a petrochemical system.

### TECHNICAL FIELD

The present invention relates to a recycling method and system, and more particularly to a recycling method and system for chemically recycling a hydrocarbon residue such as heavy oil discharged from an oil refinery process or a petrochemical process, various wastes, or organic matter.

### BACKGROUND ART

An oil refinery process is approximately defined as a process of slowly extracting light components, as a product, from crude oil while leaving heavy oil, which has a high C/H ratio, as a residue. In other words, an oil refinery process can be defined as a process of removing unnecessary carbon components as heavy oil from crude oil. Accordingly, in order to recover and reuse the residual heavy oil, which is discharged from the oil refinery process, it is important to efficiently treat unnecessary carbon components.

One of the simplest ways to treat unnecessary carbon components is to combust the carbon components. Pyrolysis and/or gasification is also effective to make efficient use of heavy oil. When heavy oil is overheated for pyrolysis and/or gasification, light components are further removed by the gasification. Only carbon components eventually remain as fixed carbon (soot and coke). Accordingly, in order to gasify the heavy oil, it is necessary to employ a gasification method capable of treating fixed carbon.

Gasification has a reaction rate lower than that of combustion. Particularly, fixed carbon is gasified at a low reaction rate. Accordingly, in order to enhance a reaction rate of gasification, a gasification process of heavy oil is generally performed at a high temperature and a high pressure. Specifically, a gasification process of heavy oil is performed at a temperature of 1000° C. to 1500° C. and a pressure of at least 1 Mpa. Generally, the temperature is increased by a direct heating method using partial oxidation in which a portion of heavy oil is reacted with oxygen. An indirect heating method cannot be employed because it is difficult to use a heat exchanger for exchanging heat in a high-temperature range.

Such a gasification method using partial oxidation produces gas which is mainly composed of H₂, CO, CO₂, and H₂O. In order to chemically recycle the produced gas, H₂ and CO may be separated and reused as needed. Alternatively, the produced gas may be used as fuel gas for a gas turbine and the like without a separation process of H₂ and CO. Since the produced gas generally has a high pressure as described above, it can be introduced into a gas turbine without further compression and is effective in a gas cleaning process or a gas refinery process prior to the introduction to the gas turbine.

With the gasification method using partial oxidation, a gasification furnace is operated at a high temperature. Accordingly, the durability of heat-resistant components such as a burner and a refractory material is lowered so as to increase costs of equipment and maintenance. Further, since the gasification process using partial oxidation is performed at a high temperature and a high pressure, it requires a high level of operation skill. Thus, an operating cost is also increased. Accordingly, in many cases, a gasification system for a high-temperature gasification process using partial oxidation is made large in size to obtain scale merit and reduce a load of an initial cost. Generally, it costs several tens of billion yen for an initial investment.

### DISCLOSURE OF INVENTION

The present invention has been made in view of the above drawbacks. It is, therefore, a first object of the present invention to provide a recycling method which can chemically recycle residual hydrocarbon heavy oil, various wastes, or organic matter at low cost.

A second object of the present invention is to provide a recycling system which can chemically recycle residual hydrocarbon heavy oil, various wastes, or organic matter at low cost.

In order to attain the first object, according to a first aspect of the present invention, there is provided a method for recycling a material such as residual hydrocarbon heavy oil, various wastes, and organic matter. In this method, a carbon component is selectively combusted in a material. The material is pyrolyzed (or thermally cracked) and gasified by using heat of combustion in the combusting process as a heat source. A pyrolysate produced in the pyrolyzing and gasifying process is supplied to an oil refinery process or a petrochemical process.

In the specification, residual hydrocarbon heavy oil is defined as oil discharged from an oil refinery process or a petrochemical process. For example, residual hydrocarbon heavy oil may comprise residual oil discharged from a. hydrocracking process such as a fluid catalytic cracking process in an oil refinery process, or cracked oil discharged from an ethylene production process. A pyrolysate is defined as a pyrolysate containing a produced gas, oil having a specific gravity of 0.7 to 1.1, and a pyrolysis residue mainly containing carbon. The produced gas may contain H₂, CO, CO₂, or light hydrocarbon such as methane, ethane, or propane. The oil having a specific gravity of 0.7 to 1.1 may comprise light oil such as a gasoline distillate, a naphtha distillate, a kerosene distillate, or a light oil distillate. The composition of such oil may vary according to types of materials, a pyrolysis temperature, types of gasifying agents, and the like.

The pyrolyzing process is defined as a reaction to decompose hydrocarbon molecules of the material by heating so as to produce hydrogen, carbon monoxide, carbon dioxide, and light hydrocarbon having three or less carbons, such as methane, ethane, or ethylene. With the pyrolyzing process, the material is decomposed into light hydrocarbon, aromatic heavy hydrocarbon such as benzene, and a carbon component. The gasifying process is defined as a reaction to produce a synthesis gas mainly containing hydrogen, carbon monoxide, and carbon dioxide from a hydrocarbon material. In the gasifying process, chemical bond of hydrocarbon of the material is cut by molecules of a gasifying agent such as oxygen, hydrogen, or steam. The material reacts with the gasifying agent to produce hydrogen, carbon monoxide, and gaseous molecules having three or less carbons, such as methane, ethane, or ethylene. The solid carbon component is converted into gaseous monoxide. Methods of producing a synthesis gas generally employ a partial oxidation method using no catalysts or a steam reforming method using a catalyst.

According to a second aspect of the present invention, there is provided a method for recycling a material such as residual hydrocarbon heavy oil, various wastes, and organic matter. In this method, a carbon component is selectively combusted in a material. The material is pyrolyzed (or thermally cracked) and gasified by using heat of combustion in the combusting process as a heat source. A pyrolysate produced in the pyrolyzing and gasifying process is cooled and cleaned. The cooled and cleaned pyrolysate is supplied to an oil refinery process or a petrochemical process.

The pyrolysate may be supplied to an atmospheric distillation process of the oil refinery process or an ethylene production process of the petrochemical process. Thus, since a cooled and cleaned pyrolysate is supplied to the oil refinery or petrochemical process, it is possible to minimize influence on the oil refinery or petrochemical process.

According to a third aspect of the present invention, there is provided a method for recycling a material such as residual hydrocarbon heavy oil, various wastes, and organic matter. In this method, a carbon component is selectively combusted in a material. The material is pyrolyzed (or thermally cracked) and gasified by using heat of combustion in the combusting process as a heat source. A pyrolysate produced in the pyrolyzing and gasifying process is separated into fractions. The fractions are supplied to an oil refinery process or a petrochemical process.

According to a fourth aspect of the present invention, there is provided a method for recycling a material such as residual hydrocarbon heavy oil, various wastes, and organic matter. In this method, a carbon component is selectively combusted in a material. The material is pyrolyzed (or thermally cracked) and gasified by using heat of combustion in the combusting process as a heat source. A pyrolysate produced in the pyrolyzing and gasifying process is cooled and cleaned. The cooled and cleaned pyrolysate is separated into fractions. The fractions are supplied to an oil refinery process or a petrochemical process.

The fractions may comprise gas, naphtha, kerosene, or light oil. The fractions may be supplied to an atmospheric distillation process of the oil refinery process or an ethylene production process of the petrochemical process. Thus, since fractions into which a pyrolysate is separated are supplied to the oil refinery or petrochemical process, it is possible to minimize influence on the oil refinery or petrochemical process.

According to a fifth aspect of the present invention, there is provided a method for recycling a material such as residual hydrocarbon heavy oil, various wastes, and organic matter. In this method, a carbon component is selectively combusted in a material. The material is pyrolyzed (or thermally cracked) and gasified by using heat of combustion in the combusting process as a heat source. A pyrolysate produced in the pyrolyzing and gasifying process is cleaned with distillate oil discharged from an atmospheric distillation process of an oil refinery process or oil into which the distillate oil has been refined. At least one of the oil used in the cleaning process and the cleaned pyrolysate is supplied to the atmospheric distillation process of the oil refinery process or a petrochemical process.

The material may include residual oil discharged from the oil refinery process or the petrochemical process. The residual oil may comprise residual hydrocarbon heavy oil discharged from an atmospheric distillation process of the oil refinery process. The residual oil may comprise residual hydrocarbon heavy oil that has been discharged from an atmospheric distillation process of the oil refinery process and flashed under a reduced pressure. The residual oil may comprise residual hydrocarbon heavy oil that has been discharged from an atmospheric distillation process or a vacuum distillation process of the oil refinery process and pyrolyzed (or thermally cracked). The residual oil may comprise residual hydrocarbon heavy oil that has been discharged from an ethylene production process of the petrochemical process. In this case, the residual oil may comprise pyrolyzed tar. The material may include wastes such as municipal solid wastes, waste, plastic, or shredder dust. Further, the wastes may comprise industrial wastes including a hydrocarbon organic compound such as tank sludge produced in an oil refinery process, a petrochemical process, or a chemical process. The material may include organic matter such as biomass. Further, the organic matter may comprise a low grade fossil material such as Orinoco tar or bituminous brown coal, coal, petroleum coke, or gas containing hydrocarbon such as coke oven gas (COG), blast furnace gas, or converter gas.

Hydrogen gas, methane gas, ethylene gas, ethane gas, propylene gas, propane gas, or steam may be used as a gasifying agent for the pyrolyzing and gasifying process. Gas recovered in the oil refinery process may be used as a gasifying agent for the pyrolyzing and gasifying process. Particles containing metal such as iron, cobalt, or ruthenium may be used as a heating medium for the pyrolyzing and gasifying process to promote hydrocarbon synthesis in the pyrolyzing and gasifying process. A substance having a desulfurization function, such as calcium oxide (CaO), calcium carbonate (CaCO₃), or calcium hydroxide (Ca(OH)₂), may be used as a heating medium for the pyrolyzing and gasifying process to promote desulfurization reaction in the pyrolyzing and gasifying process.

The pyrolyzing and gasifying process may be performed by a pyrolysis apparatus having a combustion chamber for selectively combusting the carbon component and a gasification chamber for pyrolyzing (or thermally cracking) and gasifying the material by using heat of combustion in the combustion chamber as a heat source. In this case, the pyrolysis apparatus may comprise an internal circulating fluidized-bed gasification furnace. The material may be supplied to both of the combustion chamber and the gasifying chamber of the pyrolysis apparatus.

In order to attain the second object, according to a sixth aspect of the present invention, there is provided a recycling system having a pyrolysis apparatus including a combustion section for selectively combusting a carbon component in a material, and a gasification section for pyrolyzing (or thermally cracking) and gasifying the material by using heat of combustion in the combustion section as a heat source. The recycling system has a passage for supplying a pyrolysate produced in the gasification section to an oil refinery system or a petrochemical system.

According to a seventh aspect of the present invention, there is provided a recycling system having a pyrolysis apparatus including a combustion section for selectively combusting a carbon component in a material, and a gasification section for pyrolyzing (or thermally cracking) and gasifying the material by using heat of combustion in the combustion section as a heat source. The recycling system has an oil scrubber disposed downstream of the gasification section for cooling and cleaning a pyrolysate produced in the gasification section. The recycling system also has a passage for supplying the cooled and cleaned pyrolysate to an oil refinery system or a petrochemical system.

The atmospheric distillation unit is defined as an apparatus for performing fractional distillation in an oil refinery system. The passage may be configured to supply the pyrolysate to an atmospheric distillation unit of the oil refinery system or an ethylene production system of the petrochemical system. Thus, since the oil scrubber can cool and clean the pyrolysate to be supplied to the oil refinery or petrochemical process, it is possible to minimize influence on the oil refinery or petrochemical process.

According to an eighth aspect of the present invention, there is provided a recycling system having a pyrolysis apparatus including a combustion section for selectively combusting a carbon component in a material, and a gasification section for pyrolyzing (or thermally cracking) and gasifying the material by using heat of combustion in the combustion section as a heat source. The recycling system has a fractionating tower disposed downstream of the gasification section for separating a pyrolysate produced in the gasification section into fractions. The recycling system also has a passage for supplying the fractions to an oil refinery system or a petrochemical system.

According to a ninth aspect of the present invention, there is provided a recycling system having a pyrolysis apparatus including a combustion section for selectively combusting a carbon component in a material, and a gasification section for pyrolyzing (or thermally cracking) and gasifying the material by using heat of combustion in the combustion section as a heat source. The recycling system has an oil scrubber disposed downstream of the gasification section for cooling and cleaning a pyrolysate produced in the gasification section. The recycling system also has a fractionating tower disposed downstream of the gasification section for separating the cooled and cleaned pyrolysate into fractions. The recycling system includes a passage for supplying the fractions to an oil refinery system or a petrochemical system.

The fractionating tower is defined as an apparatus for performing fractional distillation of a pyrolysate. The fractions may comprise at least one of gas, naphtha, kerosene, and light oil. The passage may be configured to supply the fractions to an atmospheric distillation unit of the oil refinery system or an ethylene production system of the petrochemical system. Thus, since the fractionating tower can separate the pyrolysate to be supplied to the oil refinery or petrochemical process, it is possible to minimize influence on the oil refinery or petrochemical process.

According to a tenth aspect of the present invention, there is provided a recycling system having a pyrolysis apparatus including a combustion section for selectively combusting a carbon component in a material, and a gasification section for pyrolyzing. (or thermally cracking) and gasifying the material by using heat of combustion in the combustion section as a heat source. The recycling system has a cleaning unit for cleaning a pyrolysate produced in the gasification section with distillate oil discharged from an atmospheric distillation unit of an oil refinery system or oil into which the distillate oil has been refined. The recycling system also has a passage for supplying at least one of the oil used in the cleaning unit and the cleaned pyrolysate to at least one of the atmospheric distillation unit of the oil refinery system and a petrochemical system.

The material may include residual oil discharged from the oil refinery system or the petrochemical system. The residual oil may comprise residual hydrocarbon heavy oil discharged from an atmospheric distillation unit of the oil refinery system. The residual oil may comprise residual hydrocarbon heavy oil that has been discharged from an atmospheric distillation unit of the oil refinery system and flashed under a reduced pressure. The residual oil may comprise residual hydrocarbon heavy oil that has been discharged from an atmospheric distillation unit or a vacuum distillation unit of the oil refinery system and pyrolyzed (or thermally cracked). The residual oil may comprise residual hydrocarbon heavy oil that has been discharged from an ethylene production system of the petrochemical system. In this case, the residual oil may comprise pyrolyzed tar. The material may include waste such as waste plastic or shredder dust. The material may include organic matter such as biomass.

Hydrogen gas, methane gas, ethylene gas, ethane gas, propylene gas, propane gas, or steam may be used as a gasifying agent in the gasification section. Gas recovered in the oil refinery process may be used as a gasifying agent in the gasification section. Particles containing metal such as iron, cobalt, or ruthenium may be used as a heating medium in the gasification section to promote hydrocarbon synthesis in the gasification section. A substance having a desulfurization function, such as calcium oxide (CaO), calcium carbonate (CaCO₃), or calcium hydroxide (Ca(OH)₂), may be used as a heating medium in the gasification section to promote desulfurization reaction in the gasification section.

The pyrolysis apparatus may comprise a combustion chamber for selectively combusting the carbon component and a gasification chamber for pyrolyzing (or thermally cracking) and gasifying the material by using heat of combustion in the combustion chamber as a heat source. In this case, the pyrolysis apparatus may comprise an internal circulating fluidized-bed gasification furnace. The recycling system may include a passage for supplying the material to both of the combustion section and the gasifying section of the pyrolysis apparatus.

According to the present invention, a pyrolysis apparatus can selectively combust a carbon component in residual hydrocarbon heavy oil, and pyrolyze (or thermally crack) and gasify residual hydrocarbon heavy oil, various wastes, and organic matter to produce a pyrolysate which has been converted into light oil. Thus, it is not necessary to gasify the carbon component or operate the pyrolysis apparatus at an increased temperature. Further, since the carbon component can be combusted, it is possible to treat superheavy oil having a large amount of asphaltene.

Further, since the pyrolysis apparatus is not operated at a high temperature, the resultant pyrolyzed gas contains hydrocarbon gas such as ethylene, propylene, and butadiene, and oil components such as naphtha, light oil, and kerosene. When the pyrolyzed gas is supplied to the oil refinery process and/or the petrochemical process, it is possible to enhance the value of the residual hydrocarbon heavy oil.

Furthermore, since the pyrolysis apparatus is not operated at a high temperature, it is not necessary to employ a direct heating method using partial oxidation. Accordingly, the resultant oil is stable because it contains no oxygen or otherwise only a small amount of oxygen. Further, produced gas contains no oxygenated compounds such as carbon dioxide or carbon monoxide or otherwise only a small amount of oxygenated compounds.

When the material includes wastes, the amount of fossil fuel consumed can be reduced so as to contribute to preservation of the global environment. Further, it is possible to improve the profitability of the recycling system through income earned from waste disposal.

In order to enhance a recycling ratio of residual hydrocarbon heavy oil, it is desirable to increase a ratio of fractions, which are produced as a product by an atmospheric distillation tower, in a pyrolysate. In a gasification furnace, Fischer-Tropsch reaction (FT reaction) may be performed for hydrocarbon synthesis.

The above and other objects, features, and advantages of the present invention will be apparent from the following description when taken in conjunction with the accompanying drawings which illustrate preferred embodiments of the present invention by way of example.

### BRIEF DESCRIPTION OF DRAWINGS

FIG. 1 is a flow diagram showing an oil refinery system including a recycling system according to a first embodiment of the present invention;

FIG. 2 is a flow diagram showing an oil refinery system including a recycling system according to a second embodiment of the present invention;

FIG. 3 is a flow diagram showing a petrochemical system including a recycling system according to a third embodiment of the present invention;

FIG. 4 is a flow diagram showing a recycling system according to a fourth embodiment of the present invention; and

FIG. 5 is a cross-sectional view showing an internal circulating fluidized-bed gasification used in a recycling system according to the present invention.

### BEST MODE FOR CARRYING OUT THE INVENTION

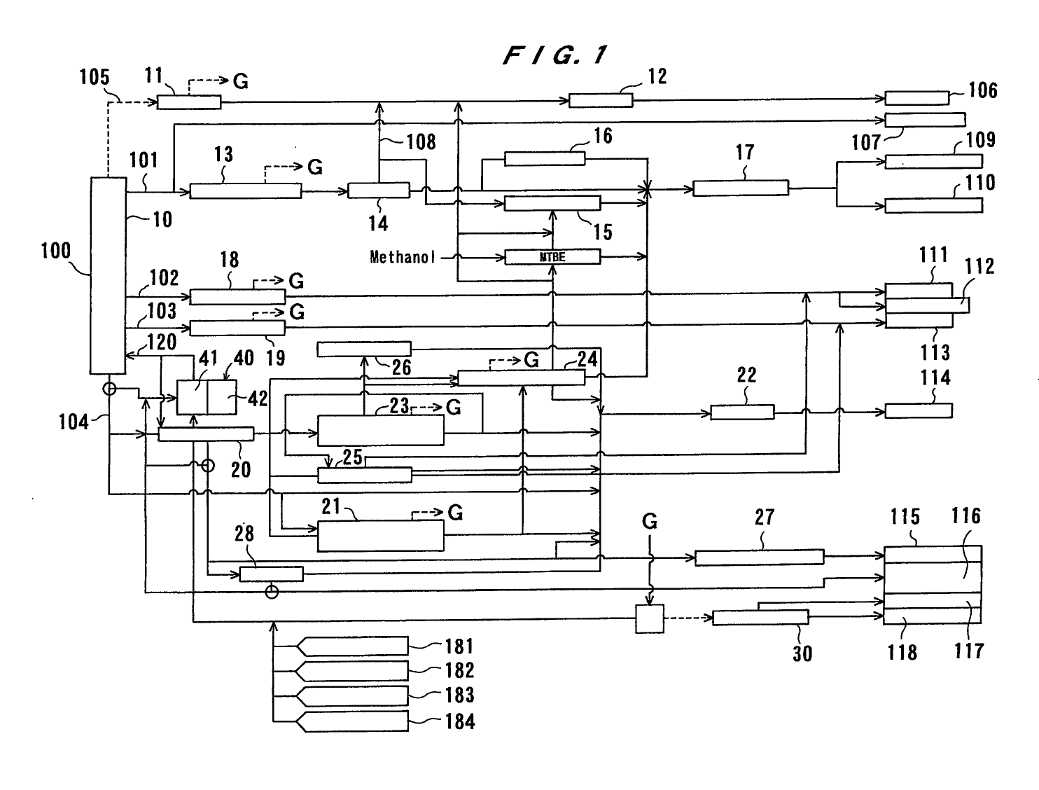

A recycling system according to embodiments of the present invention will be described below with reference to FIGS. 1 through 5. Like or corresponding parts are denoted by like or corresponding reference numerals throughout drawings, and will not be described below repetitively.

FIG. 1 shows an oil refinery system including a recycling system according to a first embodiment of the present invention. The recycling system serves to recycle residual hydrocarbon heavy oil, various wastes, and organic matter. The recycling system includes an internal circulating fluidized-bed gasification furnace 40 provided in the oil refinery system (fuel oil production system). Residual hydrocarbon heavy oil produced at respective portions of the oil refinery system is introduced into a gasification chamber 41 of the internal circulating fluidized-bed gasification furnace 40 and pyrolyzed (or thermally cracked) therein. Resultant pyrolysates 120 are returned directly to an atmospheric distillation unit 10.

Specifically, as shown in FIG. 1, the oil refinery system has an atmospheric distillation unit (atmospheric distillation tower) 10 for performing atmospheric distillation in the oil refinery process. In the atmospheric distillation unit 10, crude oil 100 is distilled under an atmospheric pressure and separated into naphtha 101, kerosene 102, light oil 103, and residual oil 104 at respective boiling ranges. Gas 105 discharged from the atmospheric distillation unit 10 is introduced into a gas recovery unit 11, which recovers light gas G (hydrocarbon gas such as hydrogen gas or methane gas), and further introduced into a LPG recovery unit 12, which recovers LPG 106 as a product.

The naphtha 101 is introduced into a naphtha hydrogenation refinery unit 13, which recovers light gas G, and further introduced into a catalytic reforming unit 14, which recovers light hydrocarbon 108. The light hydrocarbon 108 is introduced into the LPG recovery unit 12 and recovered as the LPG 106. The light hydrocarbon 108 is also supplied through an alkylation unit 15 to a gasoline compounding unit 17. The naphtha 101 that has passed through the catalytic reforming unit 14 is introduced into a benzene extraction unit 16, which extracts benzene components from the naphtha 101, and supplied into the gasoline compounding unit 17. In the gasoline compounding unit 17, gasoline is refined into automotive gasoline 109 and aviation gasoline 110. A portion of naphtha is recovered as a naphtha product 107 to be used for a petrochemical process.

The kerosene 102 distilled in the atmospheric distillation unit 10 is introduced into a kerosene hydrogenation refinery unit 18, which extracts light gas G from the kerosene 102 and produces a kerosene product 111 and jet fuel 112.

The light oil 103 distilled in the atmospheric distillation unit 10 is introduced into a gas oil hydrogenation refinery unit 19, which extracts light gas G from the light oil 103 and produces a light oil product 113.

The residual oil 104 distilled in the atmospheric distillation unit 10 is introduced into a vacuum distillation (flash) unit 20, a residual oil desulfurization (direct desulfurization) unit 21 (ARDS), and a heavy oil compounding unit 22, respectively. The residual oil 104 introduced into the vacuum distillation unit 20 is distilled under a reduced pressure at a high temperature, which is higher than the temperature of the atmospheric distillation unit 10, and then introduced into a vacuum gas oil desulfurization (indirect desulfurization) unit 23 (VGO), a (residual oil) fluid catalytic cracking unit 24, a hydrocracking unit 25, and the residual oil desulfurization unit 21, respectively. In the vacuum gas oil desulfurization unit 23, light gas G is extracted from the oil introduced thereto. Then, the oil is introduced into the heavy oil compounding unit 22. Further, a portion of the oil from the vacuum gas oil desulfurization unit 23 is introduced through a heavy gas oil desulfurization unit 26 into the heavy oil compounding unit 22. In the fluid catalytic cracking unit 24, light gas G is extracted from the oil introduced thereto. Then, the oil is introduced into the gasoline compounding unit 17. In the residual oil desulfurization unit 21, light gas G is extracted from the oil introduced thereto. Then, the oil is introduced into the heavy oil compounding unit 22. In the heavy oil compounding unit 22, the oils are compounded into a heavy oil product 114.

A portion of the residual oil discharged from the vacuum distillation unit 20 is introduced into an asphalt production unit 27 to produce asphalt 115. Another portion of the residual oil discharged from the vacuum distillation unit 20 is introduced into a decomposition unit 28, where hydrocarbon molecules of the residual oil are decomposed at a high temperature to provide light hydrocarbon molecules and petroleum pitch coke 116 as a residue.

The light gases G recovered in the gas recovery unit 11, the naphtha hydrogenation refinery unit 13, the kerosene hydrogenation refinery unit 18, the gas oil hydrogenation refinery unit 19, the fluid catalytic cracking unit 24, the vacuum gas oil desulfurization unit 23, and the residual oil desulfurization unit 21 are desulfurized in a sulfur recovery unit 30 so as to recover fuel gas 117 and sulfur 118.

In the oil refinery system of the present embodiment, the recycling system for residual hydrocarbon heavy oil includes an internal circulating fluidized-bed gasification furnace 40 having a gasification chamber 41 and a combustion chamber 42. The combustion chamber 42 serves as a combustion section for selectively combusting carbon components in materials, and the gasification chamber 41 serves as a gasification section for pyrolyzing (or thermally cracking) and gasifying the materials by using heat of combustion in the combustion section as a heat source. The materials include the residual oil 104 from the atmospheric distillation unit 10, the residual oil from the vacuum distillation unit 20, the residual oil from the decomposition unit 28, residual oils from a petrochemical system (not shown), various wastes including waste plastics 181, wastes 182, and shredder dust 183, and organic matter such as biomass 184. These materials are introduced into the gasification chamber 41 of the internal circulating fluidized-bed gasification furnace 40 and pyrolyzed (or thermally cracked) therein. Resultant pyrolysates 120 are supplied to the atmospheric distillation unit 10 or the vacuum distillation unit 20. Further, the light gases G or steam recovered at the respective portions of the oil refinery system are introduced as a fluidizing gas into the gasification chamber 41 of the internal circulating fluidized-bed gasification furnace 40.

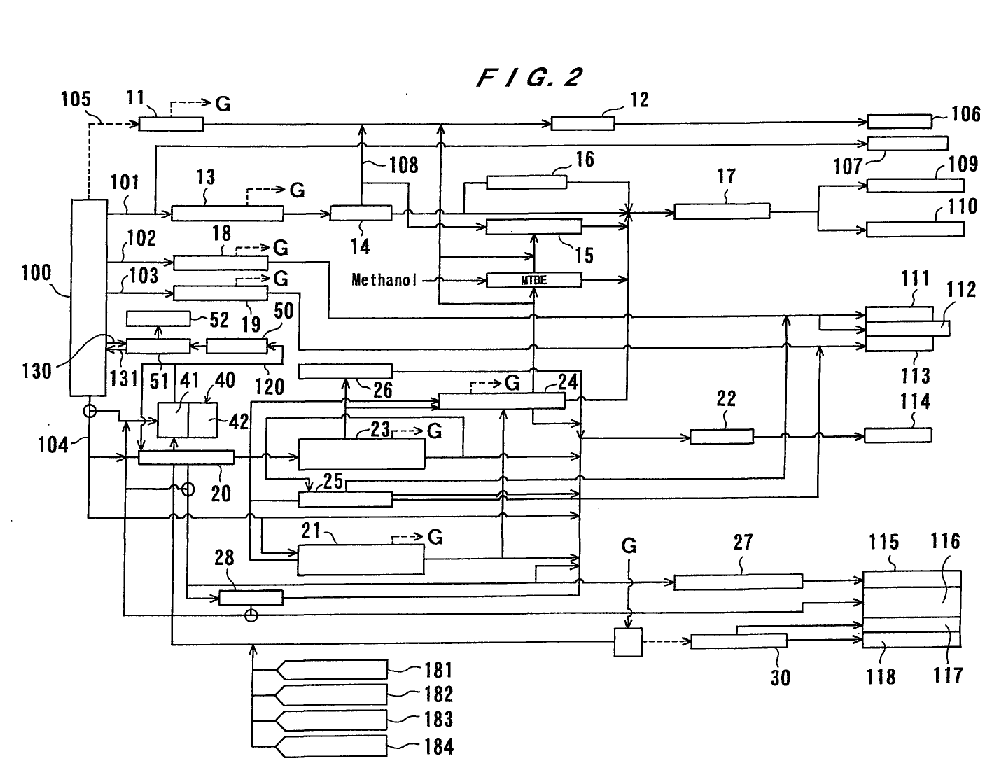

FIG. 2 shows an oil refinery system including a recycling system according to a second embodiment of the present invention. The recycling system includes an internal circulating fluidized-bed gasification furnace 40 provided in the oil refinery system as with the first embodiment. Residual hydrocarbon heavy oil produced at respective portions of the oil refinery system, various wastes, and organic matter are introduced into a gasification chamber 41 of the internal circulating fluidized-bed gasification furnace 40 and pyrolyzed (or thermally cracked) therein. Resultant pyrolysates 120 are cleaned with distillate oil from an atmospheric distillation process of the oil refinery process or oil into which distillate oil from an atmospheric distillation process of the oil refinery process is refined. The oil used for cleaning is returned directly to the atmospheric distillation process of the oil refinery process.

Specifically, as shown in FIG. 2, the recycling system for residual hydrocarbon heavy oil includes an internal circulating fluidized-bed gasification furnace 40 having a gasification chamber 41 and a combustion chamber 42 in the oil refinery system as with the first embodiment. The combustion chamber 42 serves as a combustion section for selectively combusting carbon components in materials, and the gasification chamber 41 serves as a gasification section for pyrolyzing (or thermally cracking) and gasifying the materials by using heat of combustion in the combustion section as a heat source. The materials include the residual oil 104 from the atmospheric distillation unit 10, the residual oil from the vacuum distillation unit 20, the residual oil from the decomposition unit 28, residual oils from a petrochemical process (not shown), various wastes including waste plastics 181, wastes 182, and shredder dust 183, and organic matter such as biomass 184. These materials are introduced into the gasification chamber 41 of the internal circulating fluidized-bed gasification furnace 40 and pyrolyzed (or thermally cracked) therein. Resultant pyrolysates 120 are supplied to a reforming unit 50 and reformed therein. Then, the pyrolysates 120 is introduced into a cleaning unit 51 and cleaned with distillate oil 130 withdrawn from the atmospheric distillation unit 10. The distillate oil 131 used for cleaning is returned to the atmospheric distillation unit 10. The cleaned pyrolysates 120 can be used as product gas 52.

Oil used in the cleaning unit 51 is not limited to the distillate oil 130 withdrawn from the atmospheric distillation unit 10. For example, naphtha 101, kerosene 102, or light oil 103 may be used to clean the pyrolysates 120. Further, the distillate oil 130, the naphtha 101, the kerosene 102, or the light oil 103 may be refined and used to clean the pyrolysates 120. The reforming unit 50 may be eliminated.

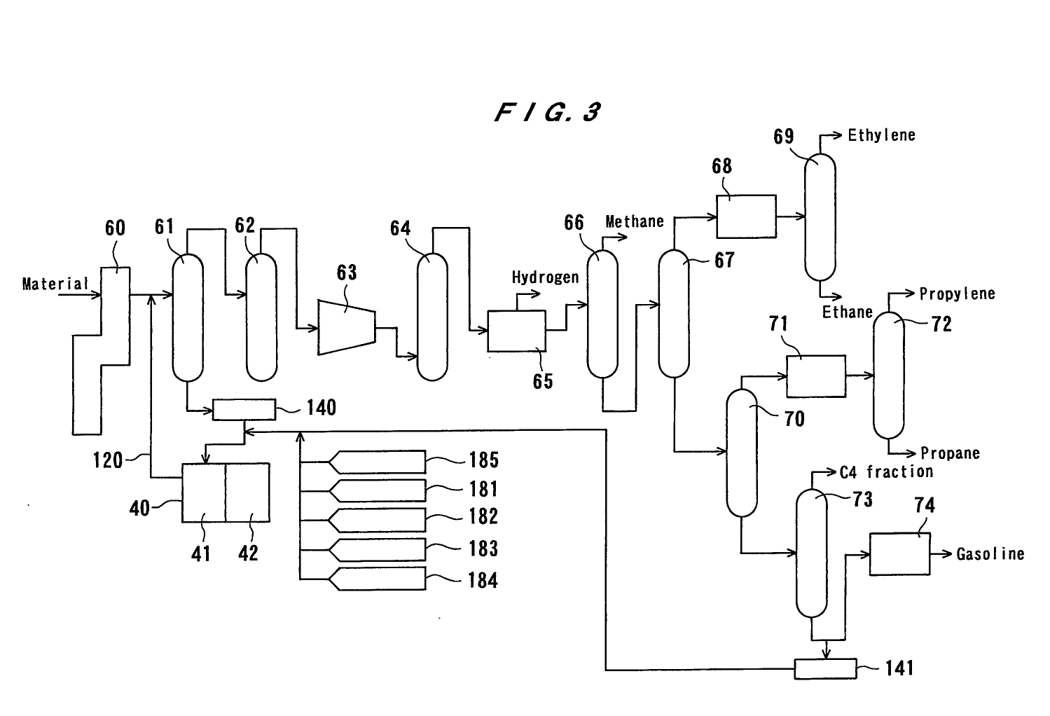

FIG. 3 shows a petrochemical system including a recycling system according to a third embodiment of the present invention. In the illustrated example, the petrochemical system includes an ethylene production system. As shown in FIG. 3, the ethylene production system has a cracking furnace 60, a gasoline fractionating tower 61, a quench tower 62, a cracked gas compressor 63, a caustic soda cleaning tower 64, a hydrogen separation unit 65, a demethanator 66, a deethanizer 67, an acetylene hydrogenation unit 68, an ethylene rectifying tower 69, a depropanizer 70, a hydrogenation unit 71, a propylene rectifying tower 72, a debutanizer 73, and a hydrogenation unit 74. The recycling system in the present embodiment includes an internal circulating fluidized-bed gasification furnace 40 having a gasification chamber 41 and a combustion chamber 42 in the ethylene production system described above.

At least one of cracked heavy oil 140 from the gasoline fractionating tower 61, a C9 fraction 141 separated at a bottom of the debutanizer 73, residual hydrocarbon heavy oil 185 from an oil refinery process or another petrochemical process (not shown), various wastes including waste plastics 181, wastes 182, and shredder dust 183, and organic matter such as biomass 184 is introduced into the gasification chamber 41 of the internal circulating fluidized-bed gasification furnace 40 and pyrolyzed (or thermally cracked) therein. Resultant pyrolysates 120 are supplied to an outlet portion of the cracking furnace 60.

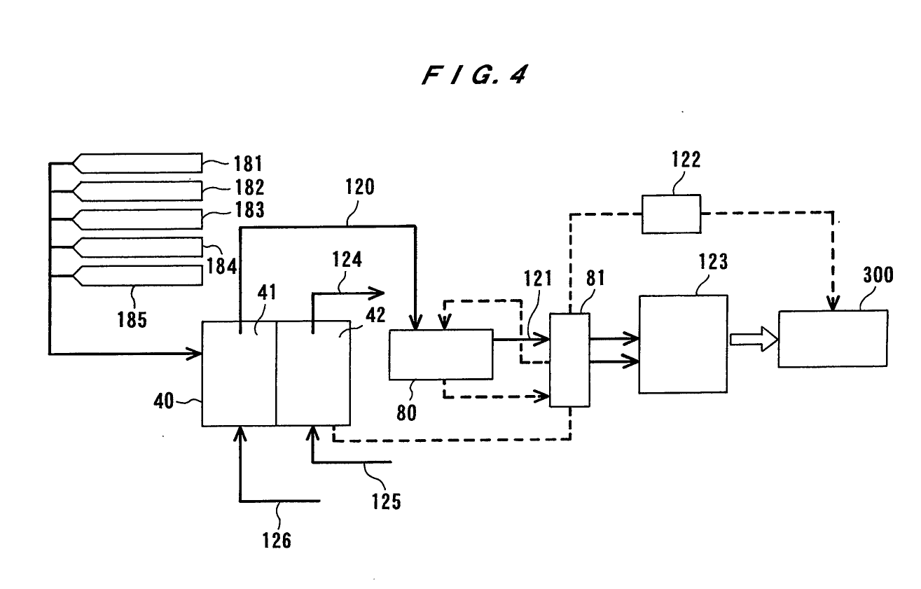

FIG. 4 shows a recycling system according to a fourth embodiment of the present invention. The recycling system is applicable to an oil refinery process as shown in FIGS. 1 and 2 and a petrochemical process (ethylene production process) as shown in FIG. 3. The recycling system includes an internal circulating fluidized-bed gasification furnace 40 having a gasification chamber 41 and a combustion chamber 42.

Residual hydrocarbon heavy oil 185 produced at respective portions of the oil refinery process or the petrochemical process, various wastes including waste plastics 181, wastes 182, and shredder dust 183, and organic matter such as biomass 184 are introduced into the gasification chamber 41 of the internal circulating fluidized-bed gasification furnace 40 and pyrolyzed (or thermally cracked) therein. Resultant pyrolysates 120 are supplied to the oil refinery process or the petrochemical process (ethylene production process).

Specifically, the recycling system includes an oil scrubber 80 disposed downstream of the internal circulating fluidized-bed gasification furnace 40 in order to minimize influence on the oil refinery or petrochemical process 300 to which the pyrolysates 120 are supplied. In the oil scrubber 80, the pyrolysates 120 are cooled and cleaned. The recycling system also includes a fractionating tower 81 disposed downstream of the oil scrubber 80. In the fractionating tower 81, the cooled and cleaned pyrolysates 121 are separated into gas 122 and fractions 123 including naphtha, kerosene, light oil, and the like. Then, the gas 122 and the fractions 123 are supplied to the oil refinery or petrochemical process 300. Off-gas or steam from the oil refinery process is supplied as a gasifying agent 126 to the gasification chamber 41 of the internal circulating fluidized-bed gasification furnace 40. Produced gas from the gasification chamber 41 or product gas from the recycling system may be supplied as a gasifying agent 126 to the gasification chamber 41 of the internal circulating fluidized-bed gasification furnace 40. Combustion air 125 is blown into the combustion chamber 42 of the internal circulating fluidized-bed gasification furnace 40.

Thus, the recycling system shown in FIG. 4 includes the internal circulating fluidized-bed gasification furnace 40 having the gasification chamber 41 and the combustion chamber 42. At least one of residual hydrocarbon heavy oil 185 from the oil refinery or petrochemical process 300, various wastes including waste plastics 181, wastes 182, and shredder dust 183, and organic matter such as biomass 184 is introduced into the gasification chamber 41 of the internal circulating fluidized-bed gasification furnace 40 and pyrolyzed (or thermally cracked) therein. Resultant pyrolysates 120 are cooled cleaned in the oil scrubber 80. The cooled and cleaned pyrolysates 121 are separated into gas 122 and fractions 123 including naphtha, kerosene, light oil, and the like in the fractionating tower 81. Then, the gas 122 and the fractions 123 are supplied to the oil refinery process 300 shown in FIG. 1 or FIG. 2 or the petrochemical process 300 shown in FIG. 3.

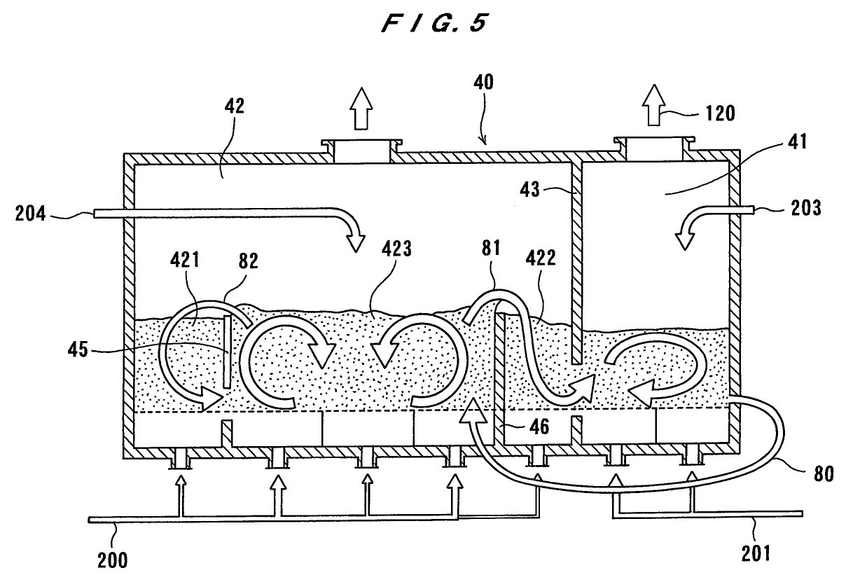

FIG. 5 shows an example of the internal circulating fluidized-bed gasification furnace 40 used in the above embodiments. As shown in FIG. 5, the internal circulating fluidized-bed gasification furnace 40 has a gasification chamber 41, a combustion chamber 42, and a partition wall 43 provided between the gasification chamber 41 and the combustion chamber 42. The combustion chamber 42 has partition walls 45 and 46 provided therein so as to form a heat recovery chamber 421, a bed material settling chamber 422, and a primary combustion chamber 423. The gasification chamber 41 and the combustion chamber 42 hold a bed material (fine particles such as sands) filled at lower portions of the gasification chamber 41 and the combustion chamber 42. As shown in FIG. 5, air 200 is supplied as a fluidizing gas for fluidizing the bed material from the bottom of the combustion chamber 42. Light gas 201 (e.g. steam or light hydrocarbon gas such as hydrogen gas, methane gas, ethylene gas, ethane gas, propylene gas, or propane gas) is supplied as a fluidizing gas for fluidizing the bed material from the bottom of the gasification chamber 41.

In the internal circulating fluidized-bed gasification furnace 40, the bed material in the gasification chamber 41 is introduced into the primary combustion chamber 423 of the combustion chamber 42 through a bed material circulation passage (not shown) as shown by an arrow 80. The bed material is increased in temperature by combustion of carbon components in the primary combustion chamber 423. The high-temperature bed material overflows the partition wall 46 into the bed material settling chamber 422 as shown by an arrow 81. The bed material in the bed material settling chamber 422 is then introduced into the gasification chamber 41 through an opening defined blow the partition wall 43. Specifically, the bed material is circulated between the gasification chamber 41 and the combustion chamber 42.

Further, the bed material in the primary combustion chamber 423 of the combustion chamber 42 overflows the partition wall 45 into the heat recovery chamber 421 as shown by an arrow 82. The bed material in the heat recovery chamber 421 is then introduced into the primary combustion chamber 423 through an opening defined below the partition wall 45. Specifically, the bed material is circulated between the primary combustion chamber 423 and the heat recovery chamber 421.

In the internal circulating fluidized-bed gasification furnace 40, materials 203 are supplied into the gasification chamber 41 at a constant rate. The materials 203 include residual hydrocarbon heavy oil discharged from the atmospheric distillation unit 10, the vacuum distillation unit 20, and the decomposition unit 28 of the oil refinery system (see FIG. 1), residual hydrocarbon heavy oil discharged from the petrochemical process, various wastes such as waste plastics, and organic matter such as biomass. Accordingly, volatile components of the materials 203 are pyrolyzed (or thermally cracked) into pyrolysates 120. The bed material containing carbon components of the materials 203 that have not been pyrolyzed in the gasification chamber 41 is moved to the combustion chamber 42 as shown by the arrow 80. The carbon components of the materials 203 are combusted in the combustion chamber 42. Heat of the combustion increases the temperature of the bed material. Then, the high-temperature bed material is introduced into the gasification chamber 41 as shown by the arrow 81 so as to contribute to pyrolysis (or thermal cracking) of the materials 203 (residual hydrocarbon heavy oil) supplied into the gasification chamber 41.

When the materials 203 to be pyrolyzed (or thermally cracked) contain more volatile components and less fixed carbon, less carbon components are introduced into the combustion chamber 42 together with the bed material as shown by the arrow 80. Accordingly, the amount of combustion in the combustion chamber 42 is reduced so that the amount of heat required in the gasification chamber 41 is not maintained. In such a case, materials 204 including residual hydrocarbon heavy oil, various wastes such as waste plastics, and organic matter such as biomass may also be supplied to the combustion chamber 42 so as to compensate for the amount of combustion in the combustion chamber 42.

As described above, the materials 203, which include residual hydrocarbon heavy oil discharged from the atmospheric distillation unit 10, the vacuum distillation unit 20, and the decomposition unit 28 of the oil refinery system (see FIG. 1), residual hydrocarbon heavy oil discharged from the petrochemical process, various wastes such as waste plastics, and organic matter such as biomass, are introduced into the gasification chamber 41 of the internal circulating fluidized-bed gasification furnace 40 and pyrolyzed (or thermally cracked) therein. The carbon components that have not been pyrolyzed (or thermally cracked) are introduced into the combustion chamber 42 together with the bed material so as to selectively combust the carbon components in the residual oil. Thus, a high-temperature atmosphere is not required to pyrolyze (or thermally crack) the materials 203 such as the residual hydrocarbon heavy oil. Accordingly, it is not necessary to employ a direct heating method using partial oxidation. The resultant oil is stable because it contains no oxygen or otherwise only a small amount of oxygen. Further, produced gas contains no oxygenated compounds such as carbon dioxide or carbon monoxide or otherwise only a small amount of oxygenated compounds.

Further, since a high-temperature atmosphere is not used to pyrolyze (or thermally crack) the materials 203, the resultant pyrolysates 120 contain hydrocarbon gas such as ethylene, propylene, and butadiene, and oil components such as naphtha and light oil. When these resultant pyrolysates 120 are supplied to the oil refinery process and/or the petrochemical process, it is possible to chemically recycle the residual hydrocarbon heavy oil, various wastes including waste plastics and shredder dust, and organic matter such as biomass.

In the above embodiments, the light gases G produced at the respective portions of the oil refinery system are used as the light gas 201 to be supplied to the gasification chamber 41 of the internal circulating fluidized-bed gasification furnace 40. However, the light gas 201 may comprise hydrogen gas (H₂), methane gas (CH₄), ethylene gas (C₂H₄), ethane gas (C₂H₆), propylene gas (C₃H₆), propane gas (C₃H₈), or steam, or a mixture thereof.

The bed material in the internal circulating fluidized-bed gasification furnace 40 may comprise particles containing metal such as iron, cobalt, or ruthenium. In such a case, hydrocarbon synthesis can be promoted in the gasification chamber 41. Alternatively, a substance having a desulfurization function, such as calcium oxide (CaO), calcium carbonate (CaCO₃), or calcium hydroxide (Ca(OH)₂), may be used as a heating medium in the internal circulating fluidized-bed gasification furnace 40.

The recycling system according to the present invention is applicable not only to an oil refinery process, but also to a petrochemical process. Further, in the above embodiments, an internal circulating fluidized-bed gasification furnace is employed as a pyrolysis apparatus having a combustion section for selectively combusting a carbon component in a material, and a gasification section for pyrolyzing (or thermally cracking) and gasifying the material by using heat of combustion in the combustion section as a heat source. However, the pyrolysis apparatus is not limited to the internal circulating fluidized-bed gasification furnace. Specifically, any apparatus may be employed as long as it has a combustion section for selectively combusting a carbon component in a material, and a gasification section for pyrolyzing (or thermally cracking) and gasifying the material by using heat of combustion in the combustion section as a heat source.

Although certain preferred embodiments of the present invention have been shown and described in detail, it should be understood that various changes and modifications may be made therein without departing from the scope of the appended claims.

### INDUSTRIAL APPLICABILITY

The present invention is suitable for use in a recycling method and system for chemically recycling a hydrocarbon residue such as heavy oil discharged from an oil refinery process or a petrochemical process, various wastes, or organic matter.

1. A recycling method comprising:
selectively combusting a carbon component in a material;
pyrolyzing and gasifying the material by using heat of combustion in said combusting process as a heat source; and
supplying a pyrolysate produced in said pyrolyzing and gasifying process to at least one of an oil refinery process and a petrochemical process.

2. A recycling method comprising:
selectively combusting a carbon component in a material;
pyrolyzing and gasifying the material by using heat of combustion in said combusting process as a heat source;
cooling and cleaning a pyrolysate produced in said pyrolyzing and gasifying process; and
supplying the cooled and cleaned pyrolysate to at least one of an oil refinery process and a petrochemical process.

3. The recycling method as recited in claim 1, wherein said supplying process comprises supplying the pyrolysate to an atmospheric distillation process of the oil refinery process.

4. The recycling method as recited in claim 1, wherein said supplying process comprises supplying the pyrolysate to an ethylene production process of the petrochemical process.

5. A recycling method comprising:
selectively combusting a carbon component in a material;
pyrolyzing and gasifying the material by using heat of combustion in said combusting process as a heat source;
separating a pyrolysate produced in said pyrolyzing and gasifying process into fractions; and
supplying the fractions to at least one of an oil refinery process and a petrochemical process.

6. A recycling method comprising:
selectively combusting a carbon component in a material;
pyrolyzing and gasifying the material by using heat of combustion in said combusting process as a heat source;
cooling and cleaning a pyrolysate produced in said pyrolyzing and gasifying process;
separating the cooled and cleaned pyrolysate into fractions; and
supplying the fractions to at least one of an oil refinery process and a petrochemical process.

7. The recycling method as recited in claim 5, wherein the fractions comprise at least one of gas, naphtha, kerosene, and light oil.

8. The recycling method as recited in claim 5, wherein said supplying process comprises supplying the fractions to an atmospheric distillation process of the oil refinery process.

9. The recycling method as recited in claim 5, wherein said supplying process comprises supplying the fractions to an ethylene production process of the petrochemical process.

10. A recycling method comprising:
selectively combusting a carbon component in a material;
pyrolyzing and gasifying the material by using heat of combustion in said combusting process as a heat source;
cleaning a pyrolysate produced in said pyrolyzing and gasifying process with distillate oil discharged from an atmospheric distillation process of an oil refinery process or oil into which the distillate oil has been refined; and
supplying at least one of the oil used in said cleaning process and the cleaned pyrolysate to at least one of the atmospheric distillation process of the oil refinery process and a petrochemical process.

11. The recycling method as recited in claim 1, wherein the material includes residual oil discharged from the oil refinery process or the petrochemical process.

12. The recycling method as recited in claim 11, wherein the residual oil comprises residual hydrocarbon heavy oil discharged from an atmospheric distillation process of the oil refinery process.

13. The recycling method as recited in claim 11, wherein the residual oil comprises residual hydrocarbon heavy oil that has been discharged from an atmospheric distillation process of the oil refinery process and flashed under a reduced pressure.

14. The recycling method as recited in claim 11, wherein the residual oil comprises residual hydrocarbon heavy oil that has been discharged from an atmospheric distillation process or a vacuum distillation process of the oil refinery process and pyrolyzed.

15. The recycling method as recited in claim 11, wherein the residual oil comprises residual hydrocarbon heavy oil that has been discharged from an ethylene production process of the petrochemical process.

16. The recycling method as recited in claim 15, wherein the residual oil comprises pyrolyzed tar.

17. The recycling method as recited in claim 1, wherein the material includes waste.

18. The recycling method as recited in claim 17, wherein the waste comprises at least one of waste plastic and shredder dust.

19. The recycling method as recited in claim 1, wherein the material includes organic matter.

20. The recycling method as recited in claim 19, wherein the organic matter comprises biomass.

21. The recycling method as recited in claim 1, further comprising using at least one of hydrogen gas, methane gas, ethylene gas, ethane gas, propylene gas, propane gas, and steam as a gasifying agent for said pyrolyzing and gasifying process.

22. The recycling method as recited in claim 1, further comprising using gas recovered in the oil refinery process as a gasifying agent for said pyrolyzing and gasifying process.

23. The recycling method as recited in claim 1, further comprising using particles containing metal as a heating medium for said pyrolyzing and gasifying process.

24. The recycling method as recited in claim 23, wherein the metal comprises iron, cobalt, or ruthenium.

25. The recycling method as recited in claim 1, further comprising using a substance having a desulfurization function as a heating medium for said pyrolyzing and gasifying process.

26. The recycling method as recited in claim 25, wherein the substance comprises calcium oxide, calcium carbonate, or calcium hydroxide.

27. The recycling method as recited in claim 1, wherein said pyrolyzing and gasifying process is performed by a pyrolysis apparatus having a combustion chamber for selectively combusting the carbon component and a gasification chamber for pyrolyzing and gasifying the material by using heat of combustion in the combustion chamber as a heat source.

28. The recycling method as recited in claim 27, wherein the pyrolysis apparatus comprises an internal circulating fluidized-bed gasification furnace.

29. The recycling method as recited in claim 27, further comprising supplying the material to both of the combustion chamber and the gasifying chamber of the pyrolysis apparatus.

30. A recycling system comprising:
a pyrolysis apparatus having a combustion section for selectively combusting a carbon component in a material, and a gasification section for pyrolyzing and gasifying the material by using heat of combustion in said combustion section as a heat source; and
a passage for supplying a pyrolysate produced in said gasification section to at least one of an oil refinery system and a petrochemical system.

31. A recycling system comprising:
a pyrolysis apparatus having a combustion section for selectively combusting a carbon component in a material, and a gasification section for pyrolyzing and gasifying the material by using heat of combustion in said combustion section as a heat source;
an oil scrubber disposed downstream of said gasification section for cooling and cleaning a pyrolysate produced in said gasification section; and
a passage for supplying the cooled and cleaned pyrolysate to at least one of an oil refinery system and a petrochemical system.

32. The recycling system as recited in claim 30, wherein said passage is configured to supply the pyrolysate to an atmospheric distillation unit of the oil refinery system.

33. The recycling system as recited in claim 30, wherein said passage is configured to supply the pyrolysate to an ethylene production system of the petrochemical system.

34. A recycling system comprising:
a pyrolysis apparatus having a combustion section for selectively combusting a carbon component in a material, and a gasification section for pyrolyzing and gasifying the material by using heat of combustion in said combustion section as a heat source;
a fractionating tower disposed downstream of said gasification section for separating a pyrolysate produced in said gasification section into fractions; and
a passage for supplying the fractions to at least one of an oil refinery system and a petrochemical system.

35. A recycling system comprising:
a pyrolysis apparatus having a combustion section for selectively combusting a carbon component in a material, and a gasification section for pyrolyzing and gasifying the material by using heat of combustion in said combustion section as a heat source;
an oil scrubber disposed downstream of said gasification section for cooling and cleaning a pyrolysate produced in said gasification section;
a fractionating tower disposed downstream of said gasification section for separating the cooled and cleaned pyrolysate into fractions; and
a passage for supplying the fractions to at least one of an oil refinery system and a petrochemical system.

36. The recycling system as recited in claim 34, wherein the fractions comprise at least one of gas, naphtha, kerosene, and light oil.

37. The recycling system as recited in claim 34, wherein said passage is configured to supply the fractions to an atmospheric distillation unit of the oil refinery system.

38. The recycling system as recited in claim 34, wherein said passage is configured to supply the fractions to an ethylene production system of the petrochemical system.

39. A recycling system comprising:
a pyrolysis apparatus having a combustion section for selectively combusting a carbon component in a material, and a gasification section for pyrolyzing and gasifying the material by using heat of combustion in said combustion section as a heat source;
a cleaning unit for cleaning a pyrolysate produced in said gasification section with distillate oil discharged from an atmospheric distillation unit of an oil refinery system or oil into which the distillate oil has been refined; and
a passage for supplying at least one of the oil used in said cleaning unit and the cleaned pyrolysate to at least one of the atmospheric distillation unit of the oil refinery system and a petrochemical system.

40. The recycling system as recited in claim 30, wherein the material includes residual oil discharged from the oil refinery system or the petrochemical system.

41. The recycling system as recited in claim 40, wherein the residual oil comprises residual hydrocarbon heavy oil discharged from an atmospheric distillation unit of the oil refinery system.

42. The recycling system as recited in claim 40, wherein the residual oil comprises residual hydrocarbon heavy oil that has been discharged from an atmospheric distillation unit of the oil refinery system and flashed under a reduced pressure.

43. The recycling system as recited in claim 40, wherein the residual oil comprises residual hydrocarbon heavy oil that has been discharged from an atmospheric distillation unit or a vacuum distillation unit of the oil refinery system and pyrolyzed.

44. The recycling system as recited in claim 40, wherein the residual oil comprises residual hydrocarbon heavy oil that has been discharged from an ethylene production system of the petrochemical system.

45. The recycling system as recited in claim 44, wherein the residual oil comprises pyrolyzed tar.

46. The recycling system as recited in claim 30, wherein the material includes waste.

47. The recycling system as recited in claim 46, wherein the waste comprises at least one of waste plastic and shredder dust.

48. The recycling system as recited in claim 30, wherein the material includes organic matter.

49. The recycling system as recited in claim 48, wherein the organic matter comprises biomass.

50. The recycling system as recited in claim 30, wherein at least one of hydrogen gas, methane gas, ethylene gas, ethane gas, propylene gas, propane gas, and steam is used as a gasifying agent in said gasification section.

51. The recycling system as recited in claim 30, wherein gas recovered in the oil refinery system is used as a gasifying agent in said gasification section.

52. The recycling system as recited in claim 30, wherein particles containing metal are used as a heating medium in said gasification section.

53. The recycling system as recited in claim 52, wherein the metal comprises iron, cobalt, or ruthenium.

54. The recycling system as recited in claim 30, wherein a substance having a desulfurization function is used as a heating medium in said gasification section.

55. The recycling system as recited in claim 54, wherein the substance comprises calcium oxide, calcium carbonate, or calcium hydroxide.

56. The recycling system as recited in claim 30, wherein said pyrolysis apparatus comprises a combustion chamber for selectively combusting the carbon component and a gasification chamber for pyrolyzing and gasifying the material by using heat of combustion in said combustion chamber as a heat source.

57. The recycling system as recited in claim 56, wherein said pyrolysis apparatus comprises an internal circulating fluidized-bed gasification furnace.

58. The recycling system as recited in claim 30, further comprising a passage for supplying the material to both of said combustion section and said gasifying section of said pyrolysis apparatus.
